(19) United States
(12) Patent Application Publication
(10) Pub. No.: US 2023/0212098 A1
(43) Pub. Date: Jul. 6, 2023

(54) Modular Methanol Upgrading Hub Methods and Systems

(71) Applicant: MX Energy Inc., Rockledge, FL (US)

(72) Inventors: Paul E. Yelvington, Rockledge, FL (US); John Anthony Dean, Scotia, NY (US); Joshua B. Browne, New York, NY (US); Edwin Yik, Orlando, FL (US)

(73) Assignee: M2X Energy Inc., Rockledge, FL (US)

(21) Appl. No.: 17/953,056

(22) Filed: Sep. 26, 2022

Related U.S. Application Data
(63) Continuation-in-part of application No. PCT/US2022/029708, filed on May 17, 2022.
Application No. PCT/US2022/029708 is a continuation-in-part of application No. PCT/US2022/029707, filed on May 17, 2022.
(60) Provisional application No. 63/248,519, filed on Sep. 26, 2021.

(51) Int. Cl. C07C 29/80

(52) U.S. Cl. CPC C07C 29/80

(57) ABSTRACT

There is provided systems and methods for aggregating and enhancing the initial materials produced from the conversion of flare gas at a flare gas source. In an embodiment the flare gas source is a hydrocarbon wellhead and the initial material is methanol and the end product is grade methanol.

This application: (i) claims under 35 U.S.C. § 119(e)(1) the benefit of the filing date of, and claims the benefit of priority to, U.S. provisional application Ser. No. 63/248,519 filed Sep. 26, 2021; (ii) claims priority to and is a continuation-in-part of PCT patent application serial number PCT/US2022/029708 filed May 17, 2022; and, (iii) claims priority to and is a continuation-in-part of PCT patent application serial number PCT/US2022/029707 filed May 17, 2022, the entire disclosure of each of which is incorporated herein by reference.

### BACKGROUND OF THE INVENTION

### Field of the Invention

The present inventions relate to new and improved methods, devices and systems for recovering and converting waste gases, such as flare gas, into useful and economically viable materials.

The term “flare gas” and similar such terms should be given their broadest possible meaning, and would include gas generated, created, associated or produced by, or from, oil and gas production, hydrocarbon wells (including conventional and unconventional wells), petrochemical processing, refining, landfills, wastewater treatment, dairies, livestock production, and other municipal, chemical and industrial processes. Thus, for example, flare gas would include stranded gas, associated gas, landfill gas, vented gas, biogas, digester gas, small-pocket gas, and remote gas.

Typically, the composition of flare gas is a mixture of different gases. The composition can depend upon the source of the flare gas. For instance, gases released during oil-gas production mainly contain natural gas. Natural gas is more than 90% methane (CH₄) with ethane and smaller amounts of other hydrocarbons, water, N₂ and CO₂ may also be present. Flare gas from refineries and other chemical or manufacturing operations typically can be a mixture of hydrocarbons and in some cases H₂. Landfill gas, biogas or digester gas typically can be a mixture of CH₄ and CO₂, as well as small amounts of other inert gases. In general, flare gas can contain one or more of the following gases: methane, ethane, propane, n-butane, isobutane, n-pentane, isopentane, n-hexane, ethylene, propylene, 1-butene, carbon monoxide, carbon dioxide, hydrogen sulfide, hydrogen, oxygen, nitrogen, and water.

The majority of flare gas is produced from smaller, individual point sources, such as a number of oil or gas wells in an oil field, a landfill, or a chemical plant. Prior to the present inventions, flare gas, and in particular flare gas generated from hydrocarbon producing wells, and other smaller point sources, was burned to destroy it, in some instances may have been vented directly into the atmosphere. This flare gas could not be economically recovered and used. The burning or venting of fare gas, both from hydrocarbon production and other endeavors, raises serious concerns about pollution and the production greenhouse gases.

As used herein unless specified otherwise, the terms “syngas” and “synthesis gas” and similar such terms should be given their broadest possible meaning and would include gases having as their primary components a mixture of H₂ and CO; and may also contain CO₂, N₂, and water, as well as, small amounts of other materials.

As used herein unless specified otherwise, the term “product gas” and similar such terms should be given their broadest possible meaning and would include gasses having H₂, CO and other hydrocarbons, and typically significant amounts of other hydrocarbons, such as methane.

As used herein unless specified otherwise, the term “reprocessed gas” includes “syngas”, “synthesis gas” and “product gas”.

As used herein unless specified otherwise, the terms “partial oxidation”, “partially oxidizing” and similar such terms mean a chemical reaction where a sub-stoichiometric mixture of fuel and air (i.e., fuel rich mixture) is partially reacted (e.g., combusted) to produce a syngas. The term partial oxidation includes both thermal partial oxidation (TPDX), which typically occurs in a non-catalytic reformer, and catalytic partial oxidation (CPDX). The general formula for a partial oxidation reaction is

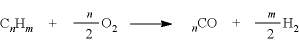

As used herein unless specified otherwise, the term “CO₂e” is used to define carbon dioxide equivalence of other, more potent greenhouse gases, to carbon dioxide (e.g., methane and nitrous oxide) on a global warming potential basis of 20 or 100 years, based on Intergovernmental Panel on Climate Change (IPCC) Fifth Assessment Report (AR5) methodology. The term “carbon intensity” is taken to mean the lifecycle CO₂e generated per unit mass of a product.

As used herein, unless specified otherwise, the terms % and mol % are used interchangeably and refer to the moles of a first component as a percentage of the moles of the total, e.g., formulation, mixture, material or product.

As used herein unless specified otherwise, the recitation of ranges of values herein is merely intended to serve as a shorthand method of referring individually to each separate value falling within the range. Unless otherwise indicated herein, each individual value within a range is incorporated into the specification as if it were individually recited herein.

Generally, the term “about” as used herein unless stated otherwise is meant to encompass the greater of a variance or range of ±10% or the experimental or instrument error associated with obtaining the stated value.

As used herein, unless stated otherwise, room temperature is 25° C., and standard temperature and pressure is 15° C. and 1 atmosphere (1.01325 bar). Unless expressly stated otherwise all tests, test results, physical properties, and values that are temperature dependent, pressure dependent, or both, are provided at standard temperature and pressure.

### Related Art and Terminology

In the production of natural resources from formations within the earth a well or borehole is drilled into the earth to the location where the natural resource is believed to be located. These natural resources may be a hydrocarbon reservoir, containing natural gas, crude oil and combinations of these; the natural resource may be fresh water; it may be a heat source for geothermal energy; or it may be some other natural resource that is located within the ground.

These resource-containing formations may be a few hundred feet, a few thousand feet, or tens of thousands of feet below the surface of the earth, including under the floor of a body of water, e.g., below the sea floor. In addition to being at various depths within the earth, these formations may cover areas of differing sizes, shapes and volumes.

Typically, and by way of general illustration, in drilling a well an initial borehole is made into the earth, e.g., the surface of land or seabed, and then subsequent and smaller diameter boreholes are drilled to extend the overall depth of the borehole. In this manner as the overall borehole gets deeper its diameter becomes smaller; resulting in what can be envisioned as a telescoping assembly of holes with the largest diameter hole being at the top of the borehole closest to the surface of the earth.

Thus, by way of example, the starting phases of a subsea drill process may be explained in general as follows. Once the drilling rig is positioned on the surface of the water over the area where drilling is to take place, an initial borehole is made by drilling a 36″ hole in the earth to a depth of about 200-300 ft. below the seafloor. A 30″ casing is inserted into this initial borehole. This 30″ casing may also be called a conductor. The 30″ conductor may or may not be cemented into place. During this drilling operation a riser is generally not used and the cuttings from the borehole, e.g., the earth and other material removed from the borehole by the drilling activity are returned to the seafloor. Next, a 26″ diameter borehole is drilled within the 30″ casing, extending the depth of the borehole to about 1,000-1,500 ft. This drilling operation may also be conducted without using a riser. A 20″ casing is then inserted into the 30″ conductor and 26″ borehole. This 20″ casing is cemented into place. The 20″ casing has a wellhead secured to it. (In other operations an additional smaller diameter borehole may be drilled, and a smaller diameter casing inserted into that borehole with the wellhead being secured to that smaller diameter casing.) A BOP (blow out preventer) is then secured to a riser and lowered by the riser to the sea floor; where the BOP is secured to the wellhead. From this point forward all drilling activity in the borehole takes place through the riser and the BOP.

For a land-based drill process, the steps are similar, although the large diameter tubulars, 30″-20″ are typically not used. Thus, and generally, there is a surface casing that is typically about 13⅜″ diameter. This may extend from the surface, e.g., wellhead and blow out preventer (BOP), to depths of tens of feet to hundreds of feet. One of the purposes of the surface casing is to meet environmental concerns in protecting ground water. The surface casing should have sufficiently large diameter to allow the drill string, product equipment such as an electronic submersible pump (ESP) and circulation mud to pass through. Below the casing one or more different diameter intermediate casings may be used. (It is understood that sections of a borehole may not be cased, which sections are referred to as open hole.) These can have diameters in the range of about 9″ to about 7″, although larger and smaller sizes may be used, and can extend to depths of thousands and tens of thousands of feet. Inside of the casing and extending from a pay zone, or production zone of the borehole up to and through the wellhead on the surface is the production tubing. There may be a single production tubing or multiple production tubings in a single borehole, with each of the production tubing endings being at different depths.

Fluid communication between the formation and the well can be greatly increased by the use of hydraulic fracturing techniques. The first uses of hydraulic fracturing date back to the late 1940s and early 1950s. In general, hydraulic fracturing treatments involve forcing fluids down the well and into the formation, where the fluids enter the formation and crack, e.g., force the layers of rock to break apart or fracture. These fractures create channels or flow paths that may have cross sections of a few microns, to a few millimeters, to several millimeters in size, and potentially larger. The fractures may also extend out from the well in all directions for a few feet, several feet and tens of feet or further. It should be remembered that the longitudinal axis of the well in the reservoir may not be vertical: it may be on an angle (either slopping up or down) or it may be horizontal. For example, in the recovery of shale gas and oil the wells are typically essentially horizontal in the reservoir. The section of the well located within the reservoir, i.e., the section of the formation containing the natural resources, can be called the pay zone.

The preceding description of upstream oil and gas production demonstrates the expense related with drilling technology and the fact that boreholes are often located in remote sites (e.g., remote onshore formations or offshore drilling platforms) and are not easily accessible by natural gas pipelines. As such, there is a need for technologies that can convert stranded gas at these well sites to liquid intermediates that can be easily brought to market. The presents inventions, among other things, provide one such way, is to convert the stranded gas to a liquid that can be further aggregated and upgraded in a hub and spoke arrangement.

As used herein, unless specified otherwise, the terms “hydrocarbon exploration and production”, “exploration and production activities”, “E&P”, and “E&P activities”, and similar such terms are to be given their broadest possible meaning, and include surveying, geological analysis, well planning, reservoir planning, reservoir management, drilling a well, workover and completion activities, hydrocarbon production, flowing of hydrocarbons from a well, collection of hydrocarbons, secondary and tertiary recovery from a well, the management of flowing hydrocarbons from a well, and any other upstream activities.

As used herein, unless specified otherwise, the term “earth” should be given its broadest possible meaning, and includes, the ground, all natural materials, such as rocks, and artificial materials, such as concrete, that are or may be found in the ground.

As used herein, unless specified otherwise “offshore” and “offshore drilling activities” and similar such terms are used in their broadest sense and would include drilling activities on, or in, any body of water, whether fresh or salt water, whether manmade or naturally occurring, such as for example rivers, lakes, canals, inland seas, oceans, seas, such as the North Sea, bays and gulfs, such as the Gulf of Mexico. As used herein, unless specified otherwise the term “offshore drilling rig” is to be given its broadest possible meaning and would include fixed towers, tenders, platforms, barges, jack-ups, floating platforms, drill ships, dynamically positioned drill ships, semi-submersibles and dynamically positioned semi-submersibles. As used herein, unless specified otherwise the term “seafloor” is to be given its broadest possible meaning and would include any surface of the earth that lies under, or is at the bottom of, any body of water, whether fresh or salt water, whether manmade or naturally occurring.

As used herein, unless specified otherwise, the term “borehole” should be given it broadest possible meaning and includes any opening that is created in the earth that is substantially longer than it is wide, such as a well, a well bore, a well hole, a micro hole, a slimhole and other terms commonly used or known in the arts to define these types of narrow long passages. Wells would further include exploratory, production, abandoned, reentered, reworked, and injection wells. They would include both cased and uncased wells, and sections of those wells. Uncased wells, or section of wells, also are called open holes, or open hole sections. Boreholes may further have segments or sections that have different orientations, they may have straight sections and arcuate sections and combinations thereof. Thus, as used herein unless expressly provided otherwise, the “bottom” of a borehole, the “bottom surface” of the borehole and similar terms refer to the end of the borehole, i.e., that portion of the borehole furthest along the path of the borehole from the borehole's opening, the surface of the earth, or the borehole's beginning. The terms “side” and “wall” of a borehole should be given their broadest possible meaning and include the longitudinal surfaces of the borehole, whether or not casing or a liner is present, as such, these terms would include the sides of an open borehole or the sides of the casing that has been positioned within a borehole. Boreholes may be made up of a single passage, multiple passages, connected passages, (e.g., branched configuration, fishboned configuration, or comb configuration), and combinations and variations thereof.

Boreholes are generally formed and advanced by using mechanical drilling equipment having a rotating drilling tool, e.g., a bit. For example, and in general, when creating a borehole in the earth, a drilling bit is extending to and into the earth and rotated to create a hole in the earth. To perform the drilling operation the bit must be forced against the material to be removed with a sufficient force to exceed the shear strength, compressive strength or combinations thereof, of that material. The material that is cut from the earth is generally known as cuttings, e.g., waste, which may be chips of rock, dust, rock fibers and other types of materials and structures that may be created by the bit's interactions with the earth. These cuttings are typically removed from the borehole by the use of fluids, which fluids can be liquids, foams or gases, or other materials know to the art.

As used herein, unless specified otherwise, the terms “formation,” “reservoir,” “pay zone,” and similar terms, are to be given their broadest possible meanings and would include all locations, areas, and geological features within the earth that contain, may contain, or are believed to contain, hydrocarbons.

As used herein, unless specified otherwise, the terms “field,” “oil field” and similar terms, are to be given their broadest possible meanings, and would include any area of land, sea floor, or water that is loosely or directly associated with a formation, and more particularly with a resource containing formation, thus, a field may have one or more exploratory and producing wells associated with it, a field may have one or more governmental body or private resource leases associated with it, and one or more field(s) may be directly associated with a resource containing formation.

As used herein, unless specified otherwise, the terms “conventional gas”, “conventional oil”, “conventional”, “conventional production” and similar such terms are to be given their broadest possible meaning and include hydrocarbons, e.g., gas and oil, that are trapped in structures in the earth. Generally, in these conventional formations the hydrocarbons have migrated in permeable, or semi-permeable formations to a trap, or area where they are accumulated. Typically, in conventional formations a non-porous layer is above, or encompassing the area of accumulated hydrocarbons, in essence trapping the hydrocarbon accumulation. Conventional reservoirs have been historically the sources of the vast majority of hydrocarbons produced. As used herein, unless specified otherwise, the terms “unconventional gas”, “unconventional oil”, “unconventional”, “unconventional production” and similar such terms are to be given their broadest possible meaning and includes hydrocarbons that are held in impermeable rock, and which have not migrated to traps or areas of accumulation.

As used herein, unless specified otherwise, the term “capital intensity” is defined as the capital cost of a chemical plant, or other capital asset engaged in production or transformation of a material, normalized by the throughput (either measured as the feed rate or production rate) of material in the plant.

As used herein, unless specified otherwise, the term “crude methanol” is defined as methanol produced in a methanol synthesis loop prior to the removal of water, dissolved gases, or other contaminants. Crude methanol often contains 5-20 wt % water, dissolved gases (e.g., 1-2 wt % CO₂) and trace contaminants (e.g., ethanol). As used herein, unless specified otherwise, the term “stabilized methanol” is defined as crude methanol that has passed through a flash operation (e.g., a single-stage flash drum) to reduce the concentration of dissolved gases and other light components. Often stabilized methanol will have <1% CO₂ and most typically about 0.5 wt % CO₂. As used herein, the terms “source methanol”, “initial methanol”, or similar terms refer to “crude methanol”, “stabilized methanol” or both. As used herein, the term “grade methanol” is defined as methanol that meets a purity standard such as the ASTM AA standard (D1152) or IMPCA methanol reference specifications.

Global Warming and Environmental Concerns

The relative harm to the environment by the release of waste gases when compared to CO₂, an established highly problematic gas, are shown FIG. 4.

The environmental impact in terms of global warming potential of methane slippage from flare gas and venting cannot be overstated. According to a 2019 International Energy Agency (IEA) report, about 200 billion cubic meter (bcm) of waste or flare gas were combusted or vented into the atmosphere in 2018. About 50 bcm of gas were vented, and about 150 bcm were combusted in flares. Combustion is intended to convert hydrocarbons to CO₂, but their peak efficiency is 98%, and that efficiency drops in the presence of wind. The combination of inefficient combustion and venting results in total CO₂e emissions of about 1.4 gigatons of CO₂, which amounts to about 2.7% of all anthropogenic sources of CO₂ per year.

This Background of the Invention section is intended to introduce various aspects of the art, which may be associated with embodiments of the present inventions. Thus, the forgoing discussion in this section provides a framework for better understanding the present inventions, and is not to be viewed as an admission of prior art.

### SUMMARY

There has been a long-standing, expanding and continuing need, for systems, devices and methods to convert otherwise uneconomic hydrocarbon-based fuel (e.g., stranded, associated, non-associated, landfill, flared, small-pocket, remote gas, wastewater treatment) to value-added, easily transported products (such as methanol, ethanol, ammonia, dimethyl-ether, F-T liquids, and other fuels or chemicals). The present inventions, among other things, solve these needs by providing the articles of manufacture, devices and processes taught, and disclosed herein.

There is provided methods and systems for upgrading methanol at a hub solves, which solves, among other things, the long-standing problem of where and how to upgrade methanol. There is a significant need for high purity methanol, which need, among others, is addressed and solved by embodiments of the present inventions, in particular, the hub and spokes embodiments.

Thus, and further, there is provided a method and system to upgrade methanol near the point of synthesis. This hub and spoke approach is more capital efficient by achieving higher utilization of upgrading capacity, at a better scale, at a location more suitable for off-taking (near railroad or major highway). In addition, non-economic gas sources may suffer from intermittency (flow that varies day-to-day, or even stops for a few days), so upgrading in a central location allows averaging across production units which permits more constant use of the plant capacity and more constant supply to off-takers.

Further, there is provided a solution to the long-standing need from a heat source for methanol processing. A significant amount of low-grade heat is required to perform the distillation process. In embodiments there is provided the thermal junction that will allow a variety of heat sources to be connected to the system. Instead of a bespoke design, this thermal junction allows a single plant design to be connected to different thermal sources. Thus, among other things, this embodiment achieves economies of scale and higher volume manufacturing.

Still further there are provided systems that are a product family that is modular and transportable allowing operational and location flexibility that is not possible with large and bespoke plants. These embodiments eliminate the cost, time and need for extensive site work such as foundations, among other things.

Still further there are provided methods for upgrading methanol using non-traditional and intensified separation technology at the spokes and hub that are tailored for distributed chemical production. These embodiments use non-thermal energy sources or combine functions of individual unit operations to reduce size, cost, energy consumption or footprint.

Thus, there is provided a system for the aggregation and enhancement of flare gas into an end product, the system including: a plurality of gas-to-liquid (GTL) systems; an initial liquid product enhancement (“IPE”) system; wherein each of the GTL systems are located a distance from the IPE system; wherein the GTL systems are in fluid communication with a flare gas source; wherein GTL systems are configured to convert the flare gas into an initial liquid product; a means for transporting the liquid initial product over each of the distances from each of the GTL systems to the IPE system; and, the IPE system configured to convert the initial liquid product into a liquid end product.

Further, there is provided these systems and methods having one or more of the following features: wherein at least one of the distances from one of the GTL systems to the IP system is different than another of the distances from another of the GTL systems to the IPE system; wherein the distances from each of the GTL systems to the IPL system are different; wherein locations of the plurality of GTL systems defines an area, and the area is from about 0.5 miles2 to about 10,000 miles2; including at least 20 GTL systems; including at least 50 GTL systems including only a single IPE system; wherein the initial liquid product has methanol; wherein the end product has methanol; wherein the initial liquid product has from about 50% to 95% methanol; wherein the initial liquid product has methanol and has less than 1% CO₂; wherein the initial liquid product has methanol having about 0.5 wt % CO₂; wherein the liquid end product has at least 99.7% methanol; wherein the liquid end product has at least 99.8% methanol; wherein the liquid end product has at least 99.85% methanol; wherein the liquid end product consists essentially of methanol, having less than 0.1 wt % water, less than 50 ppm (mg/kg) ethanol, and less than 30 ppm (mg/kg) acetone; wherein the IPE has a distillation column; wherein the IPE has a distillation column configured to remove water from the initial liquid product; wherein IPE has a distillation column configured to remove water from the initial liquid product; and wherein the distillation column has a side stream; wherein IPE has a distillation column configured to remove water from the initial liquid product; and wherein the distillation column has a dividing wall column; wherein IPE has a distillation column configured to remove water from the initial liquid product; and wherein the distillation column has a dividing wall column and a side stream; wherein IPE has a thermal junction, wherein the thermal junction has a universal heat-addition system; wherein IPE has a universal heat-addition system; wherein the means for transporting from at least one of the plurality of GTL systems has a truck, a rail car, a barge, a vessel, or a pipeline; wherein at least one of the sources of flare gas is an oil well and the GTL is in fluid communication with the oilwell; wherein at least one of the sources of flare gas is a wellhead and the GTL is in fluid communication with the wellhead; wherein at least one of sources of flare gas is a wellhead; and wherein a conduit connects the GTL system to the wellhead, whereby the GTL system is in fluid communication with the wellhead; wherein the IPE has a holding tank for receiving the initial liquid product from at least one of the plurality of GTL systems; wherein the IPE is configured to remove water from the initial liquid product; including a control system, wherein the control system is in control communication with the plurality of GTL systems; including a control system, wherein the control system is in control communication with the plurality of GTL systems and the IPE system; including a control system, wherein the control system is in control communication with one or more of the plurality of GTL systems, the IPE system, or both; wherein the GTL, the IPE or both are located off-shore.

Yet additionally, there is provided a system for the aggregation and enhancement of flare gas into an end product including methanol, the system including: a plurality of gas-to-liquid (GTL) systems, wherein the GTL systems are in fluid communication with a plurality of sources of a flare gas to thereby provide the flare gas to the GTL systems; wherein the GTL systems are configured to convert the flare gas into an initial methanol; an initial liquid product enhancement (“IPE”) system; wherein the IPE has a distillation column configured to remove water from the initial methanol, to thereby provide an end product methanol having at least 98% methanol; and, a means for conveying the initial methanol from each of the GTL systems to the IPE system.

Moreover, there is provided these systems and methods having one or more of the following features: wherein the source of the flare gas is a wellhead; wherein the plurality of GTL systems has one or more onsite GTL systems; wherein the plurality of sources of flare gas has one or more wellheads; and wherein each of the plurality of onsite GTL systems is in fluid communication with only one wellhead; wherein the plurality of GTL systems has onsite GTL systems; wherein the plurality of sources of flare gas has wellheads; and wherein at least one of the plurality of onsite GTL systems is in fluid communication with only one wellhead; wherein each of the plurality of GTL systems is an onsite GTL system; wherein the plurality of sources of flare gas are wellheads; and wherein the each of the onsite GTL systems is in fluid communication with only one wellhead; wherein locations of the plurality of GTL systems defines an area, and the area is from about 0.5 miles2 to about 10,000 miles2; including at least 20 GTL systems; including at least 50 GTL systems; including only a single IPE system; wherein the initial methanol has from about 70% to 96% methanol; wherein the initial methanol has less than 1% CO₂; wherein the initial methanol has about 0.5 wt % CO₂; wherein the end product methanol has at least 99.5% methanol; wherein the end product methanol has at least 99.7% methanol; wherein the end product methanol has at least 99.8% methanol; wherein the end product methanol has at least 99.85% methanol; wherein the end product methanol has less than 0.1 wt % water, less than 50 ppm (mg/kg) ethanol, and less than 30 ppm (mg/kg) acetone; wherein the distillation column has a side stream; wherein the distillation column has a dividing wall column; wherein the distillation column has a dividing wall column and a side stream; wherein IPE has a thermal junction, wherein the thermal junction has a universal heat-addition system; wherein IPE has a universal heat-addition system; wherein the means for conveying from at least one of the plurality of GTL systems has a truck, a rail car, a barge, a vessel, or a pipeline; wherein at least one of the sources of flare gas is a wellhead and the GTL is in fluid communication with the wellhead; wherein all of the sources of flare gas are well heads; wherein a majority of the sources of flare gas are well heads; wherein at least one of sources of flare gas is a wellhead; and wherein a conduit connects the GTL system to the wellhead, whereby the GTL system is in fluid communication with the wellhead; wherein the IPE has a holding tank for receiving the initial liquid product from at least one of the plurality of GTL systems; further including a control system, wherein the control system is in control communication with the plurality of GTL systems; further including a control system, wherein the control system is in control communication with the plurality of GTL systems and the IPE system; further including a control system, wherein the control system is in control communication with one or more of the plurality of GTL systems, the IPE system, or both; wherein the GTL, the IPE or both are located off-shore; wherein the Initial methanol or initial liquid product is a stabilized methanol; wherein the Initial methanol or initial product is a stabilized methanol; wherein the Initial methanol or initial product is a crude methanol; and wherein the GTL systems, the IPE system or both are modular systems, consisting essentially of one or more units, each of which is less than 53 feet in length.

In addition there is provided a method for the aggregation and enhancement of flare gas into an end product, the method including: placing a plurality of gas-to-liquid (GTL) systems at a plurality of locations to thereby define a GTL location for each of the plurality of GTL system; placing an initial liquid product enhancement (“IPE”) system at an IPE location; wherein each of the GTL systems is a distance from the IPE system; receiving a flow of a flare gas into one or more of the GTL systems; wherein the GTL systems receiving the flare gas converts the flare gas into an initial liquid product; and, receiving the initial liquid product from one or more of the GTL systems at the IPE system; and, the IPE system converting the initial liquid product into a liquid end product.

Still further, there is provided these systems and methods having one or more of the following features: wherein the initial liquid product has from about 25% to about 90% methanol; wherein the initial liquid product has methanol and has less than 1% CO₂; wherein the initial liquid product has methanol having about 0.5 wt % CO₂; wherein the liquid end product has at least 99.7% methanol; wherein the liquid end product has at least 99.8% methanol; wherein the liquid end product has at least 99.85% methanol; wherein the liquid end product consists essentially of methanol, having less than 0.1 wt % water, less than 50 ppm (mg/kg) ethanol, and less than 30 ppm (mg/kg) acetone; wherein a composition, a flow rate or both of the received flare gas flow varies over time; wherein a composition, a flow rate or both of the received flare gas flow varies between one or more of the plurality of GTL systems; including blending the initial liquid products from at least two of the plurality of GTL systems; and, wherein one or more of the distances is at least 0.5 miles.

Additionally, there is provided a method for the aggregation and enhancement of flare gas into an end product, the method including: receiving a flare gas into an onsite gas-to-liquid (GTL) system; converting the received flare gas into an initial methanol; receiving the initial methanol into an initial liquid product enhancement (“IPE”) system, wherein the IPE system is located at least 0.5 miles away from a location off the GTL system; and, converting the initial methanol into end product methanol having at least 98.5% methanol.

Moreover, there is provided these systems and methods having one or more of the following features: wherein the initial methanol has from about 25% to about 90% methanol; wherein the initial methanol has less than 1% CO₂; wherein the initial methanol has about 0.5 wt % CO; wherein the end product methanol has at least 99.7% methanol; wherein the end product methanol has at least 99.8% methanol; wherein the end product methanol has at least 99.85% methanol; wherein the end product methanol consists essentially of methanol, having less than 0.1 wt % water, less than 50 ppm (mg/kg) ethanol, and less than 30 ppm (mg/kg) acetone; wherein a composition, a flow rate or both of the received flare gas flow varies over time; and, including blending the initial methanol received from the onsite GTL system, with a second initial methanol received from a second GTL system.

Additionally, there is provided a method for the aggregation and enhancement of flare gas into an end product, the method including: receiving a flare gas into one or more of a plurality of onsite gas-to-liquid (GTL) system; converting the received flare gas into an initial methanol, wherein the initial methanol has crude methanol, stabilized methanol or both; wherein the composition of the initial methanol varies over time, varies between two or more of the GTL systems, or both; receiving the initial methanol from one or more of the plurality of GTL systems at a hub; wherein the hub has a tank and an initial liquid product enhancement (“IPE”) system; blending the initial methanol from at least one of the GTL systems with the initial methanol from at least another of the GTL systems, to thereby provided a blended initial methanol; wherein the IPE system is located at least 0.5 miles away from a location off one or more of the GTL systems; and, converting the blended initial methanol into an end product methanol having at least 98.5% methanol.

Furthermore, there is provided these systems and methods having one or more of the following features: wherein the blended initial methanol has from about 25% to about 90% methanol; wherein the blended initial methanol has less than 1% CO₂; wherein the blended initial methanol has about 0.5 wt % CO₂; wherein the end product methanol has at least 99.7% methanol; wherein the end product methanol has at least 99.8% methanol; wherein the end product methanol has at least 99.85% methanol; wherein the end product methanol consists essentially of methanol, having less than 0.1 wt % water, less than 50 ppm (mg/kg) ethanol, and less than 30 ppm (mg/kg) acetone; and, wherein a composition, a flow rate or both of the received flare gas flow varies over time.

### BRIEF DESCRIPTION OF THE DRAWINGS

FIG. 1 is a schematic flow diagram of an embodiment of a flare gas to grade methanol hub and spoke framework and method in accordance with the present inventions.

FIG. 2 is a schematic flow diagram of an embodiment of an initial liquid product enhancement (“IPE”) system and method in accordance with the present inventions.

FIG. 3 is a schematic flow diagram of an embodiment of an initial liquid product enhancement (“IPE”) system and method in accordance with the present inventions.

FIG. 4 is a table showing global warming potential values.

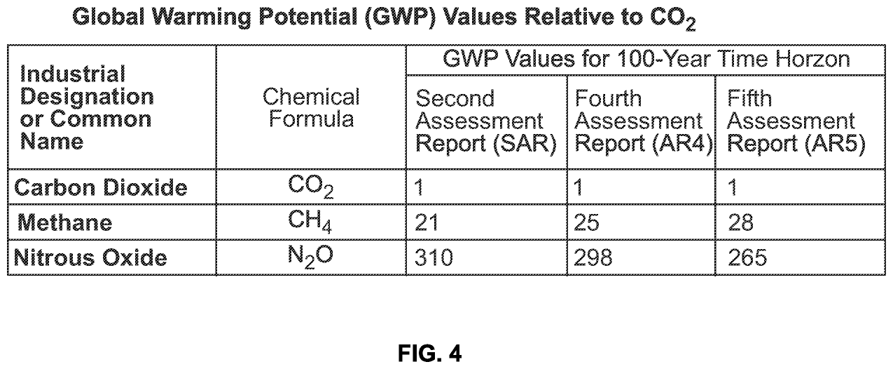

FIG. 5 is a schematic flow diagram of an embodiment of a of gas-to-liquid (“GTL”) system and method in accordance with the present inventions.

FIG. 6 is a chart showing the mole fraction of components in a distillation column for purification of stabilized methanol to grade methanol in accordance with the present inventions.

FIG. 7 is a block diagram showing an embodiment of a methanol hub and method in accordance with the present inventions.

### DESCRIPTION OF THE PREFERRED EMBODIMENTS

The present inventions generally relate to systems, devices and methods to recover in an economical fashion usable fuels from flare gas, and in particular, in an embodiment, to achieve such recovery at smaller, isolated or remote locations or point sources for the flare gas.

Embodiments of the present inventions can replace economies of scale with economies of mass manufacturing and automation, and thus, can reduce excessive operating expenses due to labor-intensive plant operations. In particular, the embodiments of the present inventions provide for reduced labor costs per product volume, autonomous, robust remote systems in the field that operate under a broad range of operating conditions and geographic locations.

In general, the present inventions relate to systems and methods for aggregating and enhancing methanol that is produced at a large number of flare gas sources. These systems, among other things, enhance the methanol produced at the flare gas sources, by among other things provide a more uniform product, improving its grade (as defined for example by ASTM, ASTM D1152-06 (2012), IMPCA), reducing its water content, or generally providing a methanol product of higher value per weight (e.g., dollars/pound) for shipping from the system, and combinations and variations of these.

In general, the present flare gas produced methanol aggregation and enhancement systems can have 2 to 10, 2 to 20, 2 to 200, 10 to 100, 20 to 200, 2 or more, 3 or more, 5 or more, 10 or more, 20 or more, 50 or more, and 100 or more sources (e.g., feed sources) of methanol that is produced from systems having individual or a collection of flare gas sources. In embodiments, these systems producing methanol from individual or a collection of flare gas sources can also have systems to enhance the uniformity, quality, value and all of these, of the methanol.

In general, embodiments of the present aggregation and enhancement system and methods involve multi-step, multi-location systems and methods for producing a “liquid end product” (such as methanol, ethanol, mixed alcohols, ammonia, dimethyl-ether, F-T liquids, and other fuels or chemicals) from multiple sources of flare gas. These methods and systems have a plurality of gas-to-liquid (“GTL”) systems. The GTL systems are in fluid communication (e.g., via pipes, valves and combinations of these) with the source of flare gas, e.g., an oil well having a wellhead. The GTL systems can be located at a flare gas source, i.e., an “onsite” GTL system, such as at, or near, a wellhead. The onsite GTL system can be less than 300 ft, less than 200 ft, less than 100 ft, less than 50 ft, and less than 20 ft from the flare gas source. These methods and systems can have an onsite GTL system at each flare gas source. These methods and systems can have a GTL system associated with several flare gas sources; e.g., one GTL system can be in fluid communication with 2, 5, 10, 2-12, 10-25 wellheads. The methods and systems can also have combinations and variations of these GTL system-flare gas source configurations.

In these general embodiments, the GTL systems produce an “initial liquid product.” In general, the initial liquid product is of lower purity than the end product, e.g., it can have a higher water concentration, as well as, other impurities. Thus, the initial liquid product can contain, preferably as its majority component, methanol, ethanol, mixed alcohols, ammonia, dimethyl-ether, F-T liquids, and other fuels or chemicals. Moreover, and typically, the purity of the initial liquid product from one or more, and potentially all of the GTL systems, can vary over time, and between and amongst the various GTL systems.

In these general embodiments, a plurality of GTL systems (e.g., 2, 4, 10, 20, 2 or more, 5 or more, 10 or more, 25 or more, 2-25, 10-50, 2-100) are associated with an initial liquid product enhancement (“IPE”) system. In this manner the initial liquid product from each of the plurality of GTL systems is transported to the IPE, where its quality, value or both, are enhanced, e.g., impurities are removed to provide a liquid end product. The GTL systems can be in fluid communication with IPE system (e.g., via pipes, valves and combinations of these), the GTL systems can be associated with the IPE system in other manners, and thus, the initial liquid product is transported from the GTL systems to the IPE systems by rail car, tanker truck, containers, totes (about 275 gallons), barrels (about 55 gallons), etc., to the IPE.

Preferably, the initial liquid product contains at least 25 wt % methanol, at least 30 wt % methanol, at least 50 wt % methanol, about 60 wt % methanol, about 75 wt % methanol, from 20 to 60 wt % methanol, from 20 to 85 wt % methanol, and from 85 to 95 wt % methanol.

Preferably, the end product is methanol, including for example, methanol having a purity of at least about 90 wt % methanol, at least about 93 wt % methanol, at least 95 wt % methanol, at least 97 wt % methanol, at least 98 wt % methanol, at least 99 wt % methanol, at least 99.5 wt % methanol, at least 99.85 wt % methanol, from about 80 to about 95 wt % methanol, from 98 to 99.9 wt % methanol, and from about 85 to about 99.5 wt % methanol and higher and lower amounts.

Preferably, the end product is methanol, including for example methanol having less than 0.1% water, less than 50 ppm (mg/kg) ethanol, and less than 30 ppm (mg/kg) acetone.

Thus, in an embodiment of the present aggregation and enhancement systems and methods, there are a plurality of flare gas sources (e.g., 2 or more, 5 or more, 10 or more, 20 or more, 25 or more, 50 or more, 100 or more). In a preferred embodiment, each of these flare gas sources has a system located onsite at the flare gas source for converting the flare gas to methanol (i.e., a gas-to-liquid system (“GTL”)). The methanol that is initially produced from the flare gas by this onsite GTL system, which is the initial liquid product, (e.g., crude methanol, stabilized methanol, source methanol, initial methanol, and combinations and variations of theses), is then transported, (e.g., flowed through a pipe, truck, drum or rail transport, as well as combinations and variation so these) to the equipment to aggregate and enhance the methanol (e.g., the hub, central process equipment, upgrade systems, enhancement system, etc.), which then improves the methanol for shipment or use. The hub typically has the IPE system, as well as, storage, piping, control valves and systems, etc., to receive, blend and process the initial methanol. In embodiments the source methanol may come from a collection of flare gas sources that are themselves aggregated, prior to or after conversion to initial methanol.

In embodiments, the initial methanol sources may be in a transfer configuration, and preferably a flow configuration (e.g., fluid communication configuration), that is a spoke and hub arrangement, with the initial methanol sources, e.g., the GTL systems, being the ends of the spokes and the enhancement system, e.g., the IPE, being at the hub. It being understood, that in the field, the arrangement may not physically look like, or be physically positioned as, a wheel, having a circular rim and spokes with a central hub. The system may also have a transfer configuration, and preferably a direct flow configuration (e.g., fluid communication configuration), that is a fish bone type with the enhancement system being at the head, the system may also have a configuration that is linear, or ladder, with the enhancement system (e.g., the IPE system, and associated tanks, values, control systems, etc.) at an end, as well as, other flow configurations. In embodiments the system may have one, two or more enhancement systems.

Flared natural gas at over 16,000 global well sites produces over 1.4 gigatons of CO₂e annually. Embodiments of the present invention utilizes that stranded, otherwise flared gas to produce economically viable, low-carbon chemicals and fuels such as methanol, hydrogen, and ammonia, thereby mitigating CO₂ emissions. For example, a system focuses on conversion of flare gas to initial methanol at the wellhead and in an oil field having a large number of wellheads.

Methanol, the simplest oxygenated hydrocarbon, is a foundational molecule that can be used for a wide variety of downstream chemicals and ultimately consumer products. An embodiment of an onsite wellhead GTL system, is a conversion platform that produces initial methanol, e.g., a stabilized methanol or crude methanol, while the primary market need is for higher grade methanol, such as ASTM AA or IMPCA grade methanol.

In general, embodiments of the present aggregation and enhancement system and methods can have a data architecture that can function under the unique and varied in-field system conditions providing, among other things, overall system remote monitoring and system evaluation, preferably in real-time, remote management of firmware and software upgrades, and reduce onboard computing requirements by offloading complex calculations and aggregations to the cloud system(s).

In general, embodiments of the present aggregation and enhancement systems and methods can have a Network Operations Center (NOC). Embodiments of the NOC have a combination of control and communication systems, processors, tools, and user interfaces that allow users to monitor, manage, and interact with individual systems and groups of systems. The items can include, for example: a customer facing dashboard, an internal team facing dashboard, and combinations and variations of these; cloud-based infrastructure that is addressable globally over the internet; databases and data models that support storing of raw data and the use of advanced mathematics for derived analytics and actionable insights. The NOC can communicate between the hub and spokes to schedule deliveries, manage inventory, predict blended product quality, estimate energy requirements, and the like.

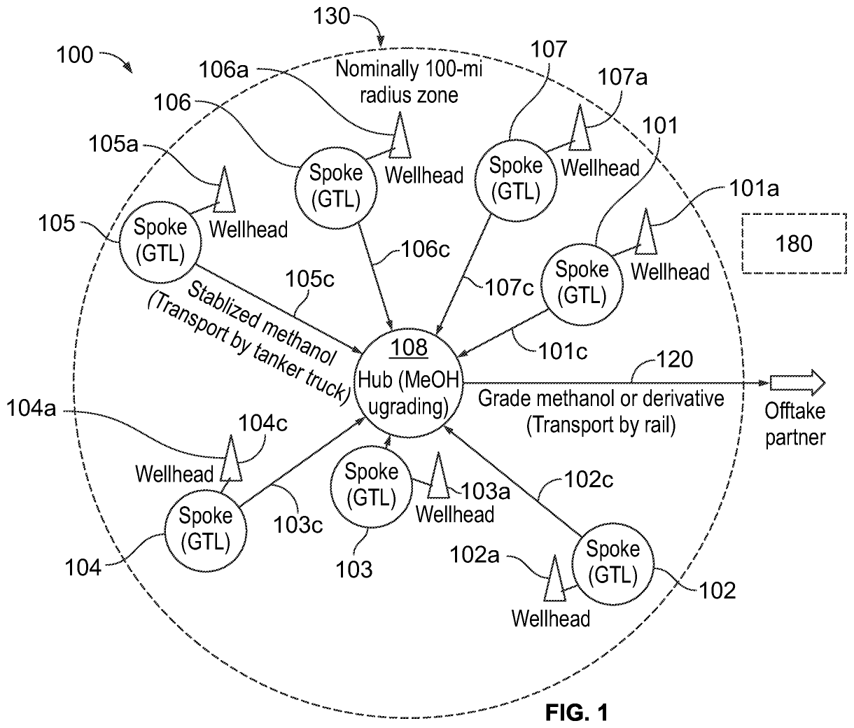

Turning to FIG. 1 there is shown an embodiment of a distributed wellhead two-stage aggregation and enhancement system 100. The distributed wellhead two-stage system 100 has a first stage, which has a plurality of GTL systems (101, 102, 103, 104, 105, 106, 107) each in fluid communication with a wellhead (101a, 102a, 103a, 104a, 105a, 106a, 107a) from which flare gas is taken and provide to the GTL systems. The system 100 has a second stage with has a hub 108 for receiving and enhancing the product, e.g., methanol, from the GTL systems. The GTL systems are located within an area 130, defined by a radius of about 100 miles (“mi”) from a central hub 108. It being understood that the area can be a square, rectangle, or other shape. The radius can be about 0.25 mi, about 0.5 mi, about 1 mi, about 5 mi, 1 mi and greater, 10 mi and greater, 50 mi and greater, 5 mi to 200 mi, 10 mi to about 100 mi and larger and smaller values. The area can be about 0.5 mi², about 1 mi², about 10 mi², about 100 mi², about 1,000 mi², about 10,000 mi², about 30,000 mi², from 0.5 mi² to 5 mi², from 1 mi² to 50 mi², from 100 mi² to 35,000 mi², and greater and smaller areas.

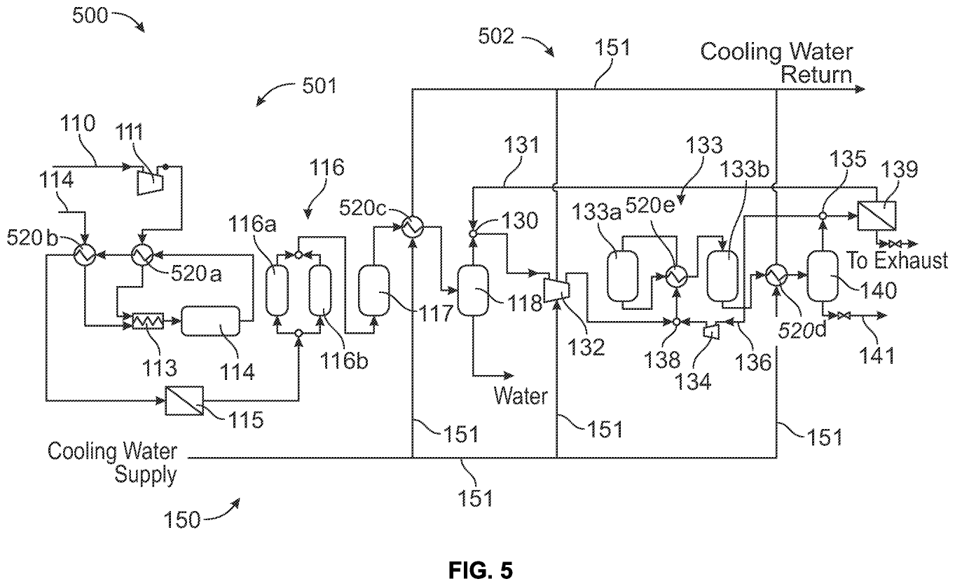

Each of the GTL systems (101, 102, 103, 104, 105, 106, 107) have equipment to collect, hold and load containers, e.g., tanker trucks, with initial methanol that is produced by the GTL systems. The GTL systems can be of the type shown in FIG. 5 and of the types generally taught and disclosed in PCT patent applications serial numbers PCT/US2022/029708 and PCT/US2022/029707 and U.S. patent application Ser. Nos. 17/746,942, 17/746,937, 17/746,927, 17/466,921 the entire disclosures of each of which are incorporated herein by reference.

The GTL systems (101, 102, 103, 104, 105, 106, 107)) provide an initial methanol. The flare gas from each of the wellheads (101a, 102a, 103a, 104a, 105a, 106a, 107a), and thus, typically to a lesser extent the initial methanol from each of the GTL systems (101, 102, 103, 104, 105, 106, 107) can vary over time in quality, composition and amount. The initial methanol is transported to the hub 108, in this embodiment the transportation is by truck, e.g., tanker truck, as shown by arrows (101c, 102c, 103c, 104c, 105c, 106c, 107c).

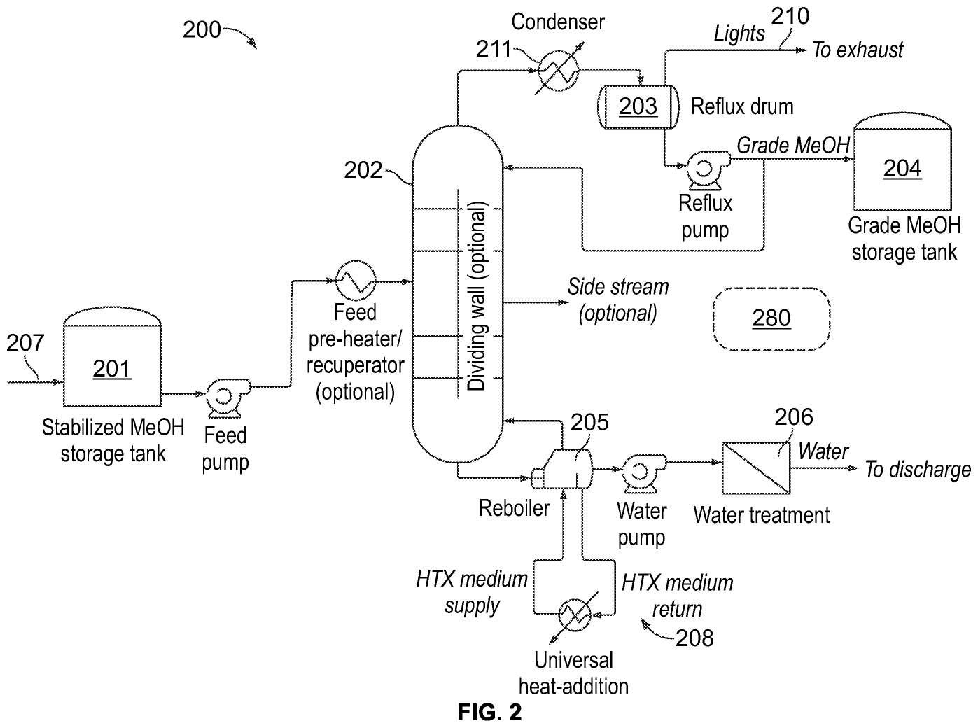

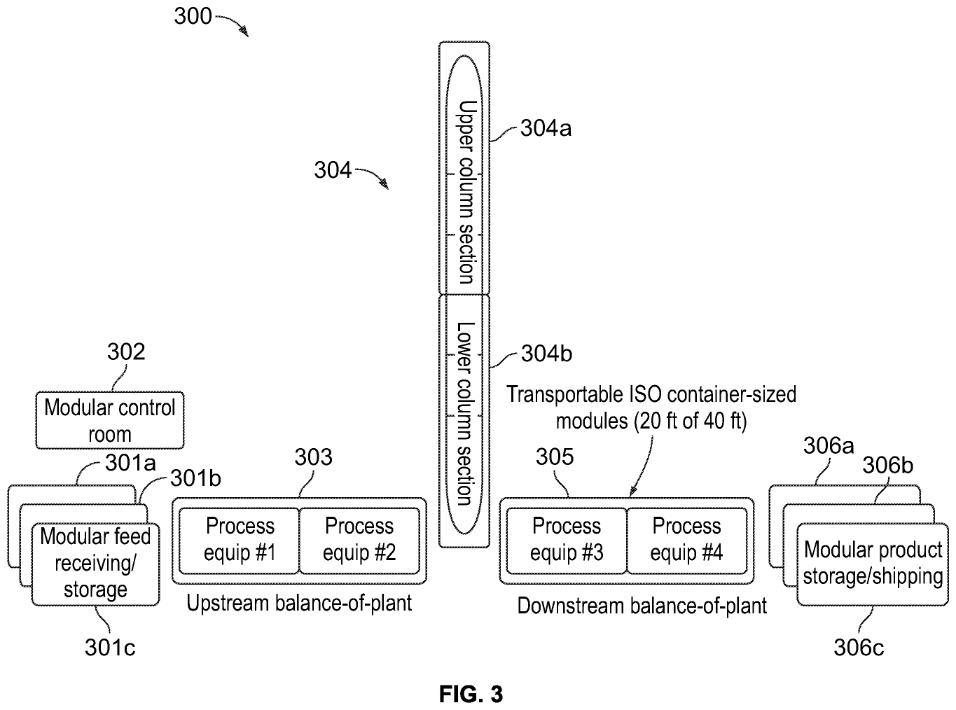

The hub 108 can have equipment for receiving, off-loading and handling, including for example, blending and storing, the initial methanol that is received from the GTL systems. The hub 108, preferably is a modular methanol upgrading (“M2Up”) hub. The hub has an IPE system that converts the initial methanol into an end product methanol. The IPE system can be, for example, a single distillation column system, a multiple distillation column system, a system of the type shown in FIG. 2, a system of the type shown in FIG. 3, and combinations and variations of these.

The end product methanol can then be transported, as shown by arrow 120, for distribution and use, by for example by tote, drum, rail, rail tank car, truck, truck tank car, pipeline and combinations and variations of these.

The distributed wellhead two-stage system 100 has an NOC or control center 180, having a control system, that receives data and input from the various sensors associated with the components of the IPE system, the hub and the GTL systems and provides control instructions to those components. In this manner the control center and the control system are in control communication with the various components and equipment of the system 100. The various components of system 100, may have local control centers and control systems, which are in control communication with control center 180 and its control system. The control system may also be distributed, in that the controllers of the various components are in control communication with controllers of the other components and with an overall systems control system. The controls system may also receive data and information from the wellheads, as well as tracking information for the trucks transporting the initial methanol. (In an embodiment the trucks transporting the initial material can be operated autonomously, semi-autonomous and remotely). The control system is configured to control the operations of the IPE system, the GTL system. The control system is further configured to take, receive and evaluate information and data about variations in the flare gas, GTL system operation, initial methanol, among other things, and based at least in part on those evaluations, adjust blending of initial methanol, staging of transport, and operation of the IPE, among other things. The control center 180 may be in a physical room, having GUIs and other user interfaces, e.g., a typical control room, (located at the hub, integral with the hub, at a remote location, and combinations and variations of these), it can be a virtual control center, e.g., cloud based, that provides the ability to display operations information and receive user input and control instruction on a GUI, e.g. a tablet or iPad, and combinations and variations of these.

Distributed wellhead systems, such a distributed wellhead two-stage system 100, address the transportation obstacle of stranded gas by densifying the gas into an easily transportable, liquid stabilized methanol intermediate (i.e., an initial liquid product, an initial methanol). Embodiments of the methanol hub, e.g., hub 108, aggregate up stabilized methanol from a collection (e.g., 25-50) of nearby wellhead GTL systems at a centralized hub to perform upgrading at a location suited for delivery to downstream markets. Preferably the siting of the hub is located at a location having good, and more preferably strong transportation infrastructure links. The hub-and-spoke flow framework, as well as other distributed and blending flow arrangements, increases uniformity of incoming feedstock for the IPE, e.g., initial methanol. The uniformity can be increased for example in terms of volume and composition, by averaging out variability at the GTL systems at the individual sources, which can be induced by flare gas variability, process variability and both. As a result, the hub-and-spoke system improves upgraded methanol production uniformity and scale (helping to secure offtake agreements and reduce capital intensity) and improves capacity factor (improving capital utilization).

In a preferred embodiment, there is a hub solution, including the IPE, that leverages modular manufacturing in a factory environment to reduce capital intensity, is transportable “over-the-road”, requires minimal site preparation and foundations, and minimal field labor for assembly and initial operation. The plant, including the IPE, operates with high levels of automation and minimal crew requirements. The present embodiments, address and overcome, among other things, challenges for the modular methanol upgrading (“M2Up”) hub, include automated operation of distributed production assets, achieving low energy intensity (and low carbon footprint) for the upgrading process, and achieving low capital intensity at a reduced scale (about 250 tones-per-day (“tpd”)), nominally 1/10^{th} scale compared to large-scale integrated methanol plants (about 3000-5000 tpd).

The system that provides the initial liquid product, e.g., initial methanol, can be a GTL system of the type shown in FIG. 5. In FIG. 5 there is shown an embodiment of a system and method for the conversion of a waste gas, e.g., flare gas, into an initial liquid product, e.g., initial methanol. The GTL system 500 has a reformer stage 501 and a synthesis stage 502. The system 500 has an air intake 110, that feeds air through into a compressor 111, which compresses the air. The compressed air is feed through heat exchanger 520a into a mixer 113. The system has a waste gas, e.g., flare gas, intake 114. The waste gas flows through a heat exchanger 520b into the mixer 113. The mixer 113, provides a predetermined mix of air and waste gas, as taught and disclosed in this specification, to a reformer 114.

The fuel-air mixture that is formed in mixer 113 is preferably rich, more preferably having an overall fuel/air equivalence ratio (0 or ER) greater than 1, greater than 1.5, greater than 2, greater than 3, from about 1.5 to about 4.0, about 1.1 to about 3.5, about 2 to about 4.5, and about 1.1 to about 3, and greater values.

It being understood that oxygen can be added to the air. And that water or steam may also be injected into the mixture of air and fuel, or to air or fuel individually. From about 1 to about 20% (molar) water can be injected, from about 10 to about 15% (molar water), from about 5 to about 17% (molar) water, more than 5% (molar) water, more than 10% (molar) water, more than 15% (molar) water, and less than 25% (molar) water, water can be injected. Following oxygen enrichment, the combustion air can have from about 21% to about 90% oxygen. “Air-breathing” reformers, and air breathing engines as used herein are understood to also include engines using air modified with the addition of water, oxygen or both.

The reformer 114 combusts the predetermined mixture of waste gas and air (e.g., flare gas and air) to form a reprocessed gas (e.g., syngas). The syngas flows through heat exchangers 520a, 520b and into a filter 115, e.g., a particulate filter.

After passing through the filter 115, the reprocessed gas (e.g., syngas) flows to a guard bed reactor assembly 116, having two guard bed reactors 116a, 116b. The guard bed reactor 116 has materials, e.g., catalysts, that remove contaminates and other materials from the syngas that would harm, inhibit or foul later apparatus and processes in the system. For example, the guard bed reactor 116 may contain catalyst or other materials to remove sulfur (e.g., iron sponge, zinc oxide or similar) and halogenated compounds.

After leaving the guard bed reactor 116, the reprocessed gas (e.g., syngas) flows to a deoxo reactor 117. The deoxo reactor 117 removes excess oxygen from the reprocessed gas (e.g., syngas) by oxidizing combustible compounds in the mixture such as methane, CO, and H₂, where the oxygen is converted to water. Catalyst for the deoxo reaction are platinum, palladium, and other active materials supported on alumina or other catalyst support materials.

The system 500 has a cooling system 150, which uses a cooling fluid, e.g., cooling water, that is flow through cooling lines, e.g., 151.

After leaving the deoxo reactor 117, the reprocessed gas (e.g., syngas) flows to heat exchanger 520c. The reprocessed gas (e.g., syngas) then flows from heat exchanger 520c to a water removal unit 118, e.g., a water knockout drum, demister, dryer, membrane, cyclone, desiccant or similar devices, where water is removed from the reprocessed gas (e.g., syngas). In general, the reprocessed gas (e.g., syngas) upon leaving unit 118 should have less than about 5% water by weight, less than about 2%, less than about 1% and less than about 0.1% water.

The overall (general) reaction for a rich fuel/air mixture to syngas is given by the equation:

ØCH₄+2[O₂+3.76N₂]→aCO+bH₂+cCO₂+dH₂O+7.52N₂

Where stoichiometric coefficients a, b, c and are determined by the chemical kinetics, conservation of atomic species, and the reaction conditions.

In addition to syngas minor constituents in the gas exiting the reformer can include water vapor, CO₂, and various unburned hydrocarbons.

After leaving unit 118, the now dry reprocessed gas (e.g., syngas) is in the synthesis stage 502. In stage 502 the now dry reprocessed gas (e.g., syngas) flows to an assembly 130. Assembly 130 provides for the controlled addition of hydrogen from line 131 into the now dry reprocessed gas (e.g., syngas). In this manner the ratio of the syngas components can be adjusted and controlled to a predetermined ratio. The hydrogen is provided from hydrogen separate 139. The ratio adjusted dry reprocessed gas (e.g., syngas) leaves assembly 130 and flow to compressor 132. Compressor 132 compresses the reprocessed gas (e.g., syngas) to an optimum pressure as taught and disclosed in this specification, for use the synthesis unit 133. Preferably, the synthesis unit 133 is a two-stage unit with a first reactor unit 133a and a second reactor unit 133b. Each reactor is a pressure vessel where process gas flows through a catalyst bed in an exothermic reaction. The catalyst bed tubes are typically immersed in a pool of cooling water at a controlled temperature and pressure. Synthesis unit 133 also has heat exchanger 520e.

The synthesis unit 133 converts the ratio adjusted dry reprocessed gas (e.g., syngas) into an initial product (e.g., initial methanol). The initial product (e.g, methanol, etc.) flows into to heat exchanger 520d. The initial product (e.g, initial methanol, etc.) flows to a collection unit 140. The collection unit 140 collects the initial liquid product (e.g, initial methanol, etc.) and flows it through line 141 for sale, holding, or further processing.

Generally, the syngas is compressed to a pressure of about 15 to about 100 bar and preferably 30-50 bar, and about 25 to about 80 bar, at least about 10 bar, at least about 25 bar and at least about 50 bar, and greater and lower pressures. The temperature of the pressurized syngas is adjusted to a temperature of about 150° C. to about 350° C. and preferably 250° C., about 200° C. to about 300° C., about 250° C. to about 375° C., greater than 125° C., greater than 150° C., greater than 200° C., greater than 250° C., greater than 350° C., and less than 400° C., and higher and lower temperatures. The pressure and temperature-controlled syngas is then feed to reactors for transforming the syngas into a more useful, more easily transportable, and economically viable product such as methanol, ethanol, mixed alcohols, ammonia, dimethyl-ether, F-T liquids, and other fuels or chemicals. In a preferred embodiment methanol is produced using the reaction of syngas to methanol, reactions for hydrogenation of CO, hydrogenation of CO₂, and reverse water-gas shift using actively cooled reactors, such as a heat-exchanged reactor or boiling water reactor, and a copper containing catalyst such as Cu/ZnO/Al₂O₃ or the like. In general embodiments of the synthesis state can use the following reactions:

CO+2H₂→CH₃OH (CO hydrogenation)

CO₂+3H₂→CH₃OH+H₂O (CO₂ hydrogenation)

CO+H₂O→CO₂+H₂ (reverse water-gas shift)

Generally, and in preferred embodiments, the characteristic length scale of the reactors used in this system are sufficiently small (e.g., micro-channel or mini-channels) that they can be shaped into unconventional shapes and topologies using new 3D printing techniques for metals and other high-temperature materials, thus allowing compact packaging and tight control over reaction conditions. Other strategies for intensification of the downstream synthesis reactions can also be considered, such as selectively removing the product from the reactor in-situ, or in a closely coupled fashion, to shift the equilibrium-limited reaction to higher conversion. This process intensification may minimize the need for large recycle streams or allow the reaction to proceed at milder conditions (e.g., lower pressure) thereby increasing process safety margins.

Typically, in reacting the syngas to form the higher value product, unreacted H₂ is also produced. The H₂ can be collected and sold, or used to power the gas turbine or a second generator to produce additional electric power.

In general, the ratio of H₂/CO in the syngas produced by the engine can be tailored to the downstream conversion process. For example, for methanol synthesis or Fischer-Tropsch (F-T) synthesis the ideal H₂/CO ratio is 2-3. For ammonia synthesis or for hydrogen production, the maximum possible H₂/CO ratio is desirable and can be enhanced by, for example, steam addition to promote the water-gas shift reaction. For ammonia and hydrogen production, the CO is not required by the downstream synthesis. As such, CO and CO₂ byproducts can be collected, sequestered, stored or utilized for other purposes.

The collection unit 140 also has a line that flows gas separated from the initial product (e.g, initial methanol, etc.) to valve 135, where it is sent to hydrogen separate 139, to a recycle loop 136 or both. Recycle loop has compressor 134 and valve 138 to feed the initial product (e.g, initial methanol, etc.) back into the synthesis unit 133. Hydrogen separation can be achieved by via membrane separation or pressure swing absorption (PSA) or the like in the hydrogen separation unit 139.

The GTL system 500 has a control system (not shown in the Figure), which is similar to the control system of NOC 180. The GTL system control system can be in direct control communication with the control center of the overall two-stage system, it can be a part of a distributed control and communication network for the overall two-stage system, and combinations and variations of this.

Enhancement system embodiments can be an IPE that utilizes a two-column direct sequence. The first column removes dissolved gases and other light components from the initial methanol, and the second column separates methanol and water. Embodiments of the enhancement system can be an IPE that utilizes a single-column arrangement with the use of a partial condenser. The single-column configuration can be used to eliminate one distillation column (including reboiler and condenser). Combinations and variations of these may also be utilized as the methanol enhancement systems.

Turning to FIG. 2 there is shown a schematic of an IPE system 200. This IPE systems can be used with any of the embodiments of a distributed wellhead two-stage system, including, for example, as part of the hub 108, and preferably a M2Up hub, in the distributed wellhead two-stage system 100. The IPE system 200 receives initial methanol, as shown by arrow 207, for example from a plurality of GTL systems, which are associated with wellheads, and configured in a spoke and hub, fishbone, ladder, linear, or other configurations. The initial methanol is placed in holding tank 201, where it can be blended or mixed with other initial methanol batches. Although one tank 201 is shown, it is understood that multiple tanks can be used for storage and blending. The initial methanol is pumped from tank 201 by a pump and through an optional pre-heater, to provide a pre-heated initial methanol. The pre-heated initial methanol is feed into the distillation column 202, where it is distilled. Purified methanol and light weight contaminates are removed from the top of distillation column 202 and feed to a condenser 211, where they are condensed to provide a distillate. The distillate is then feed to a reflux drum 203, where lightweight materials (e.g., dissolved CO₂, CO, Ar, and N₂) are removed and feed to an exhaust through line 210. High grade end product methanol, preferably greater than 99.85% pure, is pumped to storage tank 204.

The bottoms stream is taken off from the bottom of the distillation column 205 and feed to a reboiler 205, having a thermal junction system 208, having a universal heat addition system, and a heat exchange of the supply line and a heat exchanger on the return line. The water is then pumped to an optional water treatment system 206 and discharged. The thermal junction is configured to have a universal heat-addition system. The universal heat addition system is configured to accept process heat from gas-fired burners, waste-gas burners, liquid fired burners, electric heaters, solar thermal heaters, geothermal heaters, and the like. The universal heat addition system is configured for flexible, plug-and-play exchange of heat sources. The electric power for electrical process heaters can optionally be provided by renewable power sources such as solar or wind generators.

Grade methanol (e.g., ASTM AA or IMPCA) standards include specifications for trace components, such as ethanol, which cannot exceed 50 ppm (mg/kg). This maximum ethanol concentration can be difficult to achieve via distillation because ethanol and methanol are a close-boiling pair with similar relative volatility. The distillation column 202 can optionally have a side stream, as shown in FIG. 2 that separates out the middle-boiling components such as ethanol. In a preferred embodiment, the distillation column 202 is a dividing wall column (DWC) that includes an internal dividing wall as shown in FIG. 2. The DWC emulates a sequence of columns that includes a pre-fractionator, often referred to as a Petlyuk column arrangement. The DWC is capable of achieving a crisp separation of ternary (and more complex) mixtures such as methanol, ethanol, and water. The DWC is an improvement on columns with a side stream only because the feed cannot bleed into the side stream. Furthermore, the cost of the DWC is less than the traditional multi-column Petlyuk arrangement, needing only a single column with specialized internals.

System 200 has a control system 280, which is similar to the control system of control center 180. The control system 280 can be the control system for the control center of an overall two-stage system, it can be in direct control communication with the control center of the overall two-stage system, it can be a part of a distributed control and communication network for the overall two-stage system, and combinations and variations of these. The control center can issue requests to redeploy the GTL systems or adjust their operating conditions based on product volume or quality at the IPE.

Turning to FIG. 3 there is shown a schematic of a M2Up system having an IPE. The M2Up system 300 has eleven modular units, which can be separated for shipping and then assembled into the M2Up system. Preferably, each modular unit can fit on an over-the-road conventional truck bed or trailer, e.g., a semi-trailer from about 20 ft to about 40 ft in length or up to about 53 ft in length. (Understanding that longer trailers could be used, but it is preferable to not used oversized load trailers and equipment.)

The system 300 has three initial methanol receiving and storage units 301a, 301b, 301c. These units 301a, 301b, 301c when assembled into the system receive the initial methanol from the GTL systems. When assembled into the system, units, 301a, 301b, 301c are in fluid communication with each other; and can among other things, receive, store, and blend the initial methanol. These units 301a, 301b, 301c, when assembled into the system, are in fluid communication with the upstream processing unit 303.

The upstream processing unit 303 has a first assembly of process equipment and a second assembly of process equipment. Unit 303 when assembled into the system is in fluid communication with distillation column 304, which may be composed of two units, an upper column section 304a and a lower column section 304b. The distillation column 304 is in fluid communication with downstream unit 305, when the units are assembled into the system, with the distillate and the water being feed to separate equipment of downstream equipment unit 305. Unit 305 has a third assembly of process equipment and a fourth assembly of process equipment. The unit 305, when assembled into the system, is in fluid communication with three storage and handling units, 306a, 306b, 306c, where the upgraded end product methanol is stored and transferred for shipment or use. The system 300 has a control room unit 302, having a control system, that when assembled into the system, is in control communication with the other equipment of the system. Fewer or more than three methanol and storage units, for both initial methanol and upgraded methanol, are also contemplated based on the volume of the units and throughput of the IPE.

In an embodiment the system 200 is configured into mobile units along the lines of the embodiment of FIG. 3, for shipping and assembly at a hub. In preferred embodiments, the sizing of the GTL systems and the hub provides for a large majority of the market for wellhead flare gas sources to be served with only two product sizes, e.g., capacities, of 1 ton/day to 10 ton/day initial product for each GTL system and capacities of 125 ton/day to 250 ton/day end product production for the hub), where multiples of these units can be deployed at a site in parallel. This sizing provides for the further benefit of standardized, mass-produced systems. The skids can have flexible, reusable connections for gases, liquids, signals, power (electric, hydraulic or pneumatic). Configuration is modular and skid-mounted such that it can be assembled in a factory setting. Optionally, the skid can have integrated wheels and navigation lights such that they can be transported by truck or containerized for multi-modal transport by road, ship or rail. In an embodiment, the GTL spokes includes a single-stage flash system, which as the pressure is reduced from synthesis pressure (nominally 50 bar) to near ambient, reduces the need for separation of dissolved gases and other light components at the hub. The loss of methanol in the lights stream from the flash drum is considered negligible and aligns with the design philosophy to favor simplicity and robustness over absolute efficiency, for this embodiment. Operational flexibility is an advantage of the embodiments of the present systems and methods to improve turndown ratio for methanol distillation, especially during build-out of a region.

In embodiments one or more, or all of the, the initial sources of methanol in the system may provide a reprocessed gas, which is then converted to methanol, or other products, at the enhancement system, e.g., the hub.

In embodiments of the present enhancement systems the initial sources, e.g., the spokes of a hub configuration, have one or more of a gas turbine, a reciprocating engine, or both, to produce reprocessed gas, preferably syngas, are preferred under certain circumstances (such as magnitude of wellhead flow), as they provide advantages over embodiments using reciprocating engines to produce syngas.

In embodiments the sources of the waste gas (e.g., flare gas from a wellhead), can be adjacent to the enhancement system, e.g., the hub, or from 50 feet to 5 miles away, more than 100 feet, more than 1,000 feet, more than 2,000 feet, from 50 feet to 10,000 feet, less than 2 miles, less than 1 mile and greater and smaller distances.

The system can have waste gas sources at the same or different distances from the enhancement system. Similarly, the enhancement system can also be similarly located near sources of waste heat for processing the methanol in the enhancement system.

The following examples are provided to illustrate various embodiments of the present flare gas conversion processes and systems. These examples are for illustrative purposes, may be prophetic, and should not be viewed as, and do not otherwise limit the scope of the present inventions.

### EXAMPLES

### Example 1

An aggregation and enhancement system having one or more of the following configurations.

| Description | Embodiment | |
|---|---|---|
| Process Scale | Two scales for production of grade methanol: | |
| | 1) | 125 ton/day, and |
| | 2) | 250 ton/day. |
| | Both scales are modular and transportable. | |
| Heat Source | Sourcing process heat for distillation: | |
| | 1. | Thermally integrated hub that uses waste heat |
| | | from an adjacent flare-gas-methanol system, |
| | 2. | Thermally integrated hub uses non-traditional heat |
| | | sources such as low-grade heat from an adjacent |
| | | facility, solar thermal heat, etc., |
| | 3. | Thermally isolated hub that uses a gas-fired heater. |
| | | For example, and in particular, a standardized |
| | | universal heat-addition interface that can accept |
| | | heat from a variety of inside the battery limits |
| | | (ISBL) and outside the battery limits (OSBL) |
| | | sources. |
| | 4. | Process heat from electrical source, either direct |
| | | electrical heating or heat from a heat pump |
| | | powered by electricity. |
| Heat | Heat integration options to reduce the heat duty of the | |
| Integration | distillation column including: | |
| | 1. | Recuperating of heat from an electric generator |
| | | in a thermally and electrically isolated hub |
| | | that produces its own power and heat, |
| | 2. | Recuperating of heat from the condenser to |
| | | pre-heat the column feed. |
| Column Design | Distillation column design embodiments including: | |
| | 1. | Single column and two-column direct sequence |
| | 2. | Packed column and tray column, |
| | 3. | Feed stage location, |
| | 4. | Number of stages, |
| | 5. | Partial condenser designs, |
| | 6. | Hybrid and intensified distillation options, |
| | 7. | Direct air-cooled condenser and indirect |
| | | cooling loop. |
| OPEX | Design embodiments for: | |
| Reduction | 1. | Semi-autonomous operation, |
| | 2. | Optimized maintenance schedules, |
| | 3. | Reduction in energy costs. |
| Module | Packaging embodiments for: | |
| Packaging | 1. | Modules sized for ISO shipping container |
| | | size/weight limitations, |
| | 2. | Modules sized for US commercial over-the-road |
| | | trucking size/weight limitations. |

### Example 2

An aggregation and enhancement system having one or more of the following configurations.

| Item | Parameter | Requirement | | | Details |
|---|---|---|---|---|---|
| Plant | Grade methanol | 1) 125 ton/day | | | |
| Capacity | production rate | 2) 250 ton/day | | | |
| (Output) | | | | | |
| Inlet | Stabilized | Methanol 92.04% | | | |
| | methanol | Water 6.74% | | | |
| | composition (w/w) | Carbon dioxide 0.84% | | | |
| | | Ethanol 0.37% | | | |
| | | Nitrogen | 69 | ppm | |
| | | Carbon monoxide | 24 | ppm | |
| | | Argon | 6 | ppm | |
| | | Hydrogen | 3 | ppm | |
| Outlet | Upgraded | Methanol | 99.85% | min | Per ASTM D1152 |
| | methanol | Water | 0.1% | max | (Grade AA) and |
| | composition (w/w) | | | | IMPCA¹ |

| | | | |
|---|---|---|---|
| Other Outlet | Upgraded | Meets ASTM D1152 (Grade AA) | |
| | methanol | and IMPCA specs | |
| | properties | | |
| Emissions | Air, water, noise | Compliant with local, national | |
| | | standards | |
| Safety | 1) Occupational | 1) Compliant with local, national | |
| | safety | standards | |
| | 2) Process safety | 2) Considers Inherently Safer | |
| | | Design principles | |
| Ambient | | −20 to 45° C.; 0 to 100% RH | |
| Maintenance | Uptime | 90% min | 328 days/yr min |
| Product Life | Years | 16 years min | Plant useful life |
| Module | 1) U.S. over-the- | 1) 80,000 lb gross weight (truck- | 1) U.S. DOT FHWA |
| Transport | road | trailer combo); 53′ L × 8.5′ W × | (assume 35,000 lb |
| | 2) Int'l intermodal | 13.5′ H (incl. trailer) | unladen vehicle |
| | | 2) ISO 20-ft, 40-ft, or 40-ft “high | weight) |
| | | cube” compliant | 2) Meets ISO |
| | | | capacity and |
| | | | external dimensions |
| Turndown | Capacity factor | Operable down to 50% of rated | 2:1 turndown ratio |
| | | capacity | |

### Example 3

The enhancement system for use as for example the hub of a spoke and hub flow configuration, is the Modular Methanol Upgrading (M2Up) shown in FIG. 2.

### Example 4

The enhancement system for use, as for example the hub of a hub and spoke flow configuration, is the Modular Methanol Upgrading (M2Up) shown in FIG. 3.

### Example 5

An aggregation and enhancement system where the spokes are any of the individual methanol synthesis systems disclosed and taught in FIG. 5 and of the types generally taught and disclosed in PCT patent applications serial numbers PCT/US2022/029708 and PCT/US2022/029707 and U.S. patent application Ser. Nos. 17/746,942, 17/746,937, 17/746,927, 17/466,921, the entire disclosures of each of which are incorporated herein by reference, that are sized for a given (flare) site. The GTL output is crude methanol (70-95% methanol with the balance mostly), and the output rate will be subject to the inherent variable flow per day and composition at each site.

The hub takes crude methanol and produces high quality (ASTM AA grade or similar) methanol that the market demands. The hub runs at a steady level compared to the spokes. In other words, the system provides for the conversion of variable, and highly variable, non-economic (flare) gas into a reliable source of grade methanol production suitable for downstream supply chains.

The connection between the hub and the spokes is a transportation network of tanker trucks. In one embodiment, these trucks will run on the output of the methanol produced within the network.

### Example 6

The systems of any of the embodiments where the components are configured and the system is operated in a net carbon neutral manner.

### Example 7

The systems of any of the embodiments where the components are configured and the system is operated in a manner that reduces the total carbon output from the waste gas sources.

### Example 8

The embodiments of the present systems are operated in a carbon neutral-to-negative manner, producing and releasing less than or equal to zero CO₂e from a lifecycle perspective.

### Example 9

Embodiments the present systems produce an end product (e.g., high-grade methanol) that provides a net negative CO₂e for the process and the making of the end product. Thus, in preferred embodiments the process and resultant end product (e.g., methanol) has from about −40 kg CO₂e to ˜130 kg CO₂e, less than ˜20 kg CO₂e, less than ˜40 kg CO₂e, less than ˜60 kg CO₂e, less than ˜100 kg CO₂e and less than −130 kg CO₂e per kg of downstream product (e.g, liquid methanol). It should be noted that the typical CO₂e for methanol produced from natural gas is 2.1 kg CO₂e per kg methanol (based on 45 kg CO₂e per MMBTU methanol, 1,040 btu/scf natural gas, and 0.8 kg natural gas per m³). CO₂e (carbon dioxide equivalent) is based on a 20-year time horizon global warming potential for methane, based on the IPCC AR5 estimate for methane, and is 85× the global warming potential of CO₂.

### Example 10

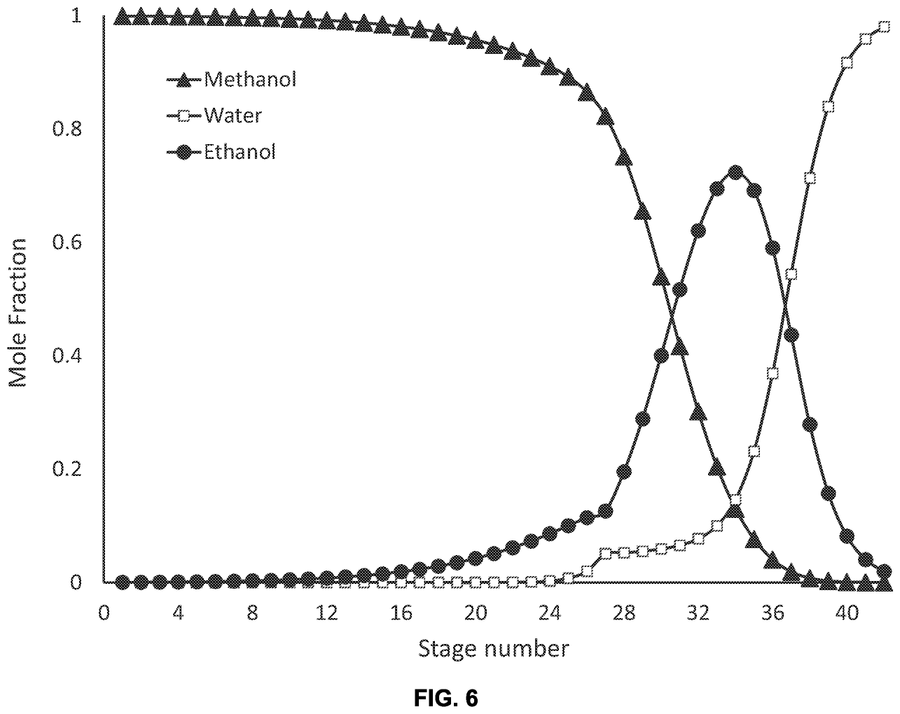

The embodiments of the present system are operated according to the schematic shown in FIG. 2 with the stabilized methanol composition shown in EXAMPLE 2. A distillation column with 42 equilibrium stages is simulated using established equations-of-state and thermodynamic property correlations. The resulting profiles of methanol, ethanol, and water mole fractions along the length of the column are shown in FIG. 6. Stage 1 is at the top of the column (distillate) and Stage 42 is at the bottom of the column (bottoms). The column uses a reboiler and partial condenser. The feed stage is on Stage 27. As shown, there is a peak of ethanol at about Stage 34 indicating that this would be well suited for a side stream at that stage. Furthermore, this indicates that a DWC configuration would be able to be likely to achieve even a crisper separation of the mixture.

### Example 11

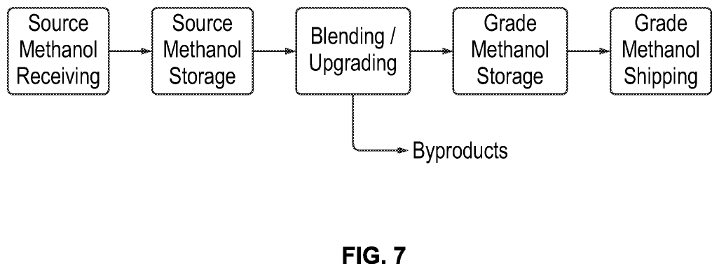

The embodiments of the present system are operated according to the schematic shown in FIG. 7, which shows the principal components of the M2Up hub. In this arrangement, the blending/upgrading step separates source methanol into grade methanol and byproducts (e.g., water). In this embodiment the upgrading step could be a membrane module, ultrasonic separator, absorption column, adsorption column or other non-thermal separation equipment.

### Example 12

The embodiments of the present systems can be configured for offshore operation. Thus, embodiments of the GTL systems are positioned on individual oil rigs, with the hub being centrally location on a rig, a vessel, a platform, a barge or on shore. Initial methanol can be transported to the hub by barge or pipe line.

Embodiments of the present systems can be configured along the lines of a Floating Production Storage and Offloading (FPSO) unit. In embodiments of the present systems, the GTL systems, the IPE system and both, can be located on the FPSO. In embodiments, the IPE can be on a ship, barge or vessel that is brought to the FPSO (having GTL systems) to upgrade the methanol off source for direct transport to a customer or user.

It is noted that there is no requirement to provide or address the theory underlying the novel and groundbreaking production rates, performance or other beneficial features and properties that are the subject of, or associated with, embodiments of the present inventions. Nevertheless, various theories are provided in this specification to further advance the art in this important area, and in particular in the important area of hydrocarbon exploration, production and downstream conversion. These theories put forth in this specification, and unless expressly stated otherwise, in no way limit, restrict or narrow the scope of protection to be afforded the claimed inventions. These theories many not be required or practiced to utilize the present inventions. It is further understood that the present inventions may lead to new, and heretofore unknown theories to explain the conductivities, fractures, drainages, resource production, chemistries, and function-features of embodiments of the methods, articles, materials, devices and system of the present inventions; and such later developed theories shall not limit the scope of protection afforded the present inventions.

The various embodiments of devices, systems, activities, methods and operations set forth in this specification may be used with, in or by, various processes, industries and operations, in addition to those embodiments of the Figures and disclosed in this specification. The various embodiments of devices, systems, methods, activities, and operations set forth in this specification may be used with: other processes industries and operations that may be developed in the future: with existing processes industries and operations, which may be modified, in-part, based on the teachings of this specification; and with other types of gas recovery and valorization systems and methods. Further, the various embodiments of devices, systems, activities, methods and operations set forth in this specification may be used with each other in different and various combinations. Thus, for example, the configurations provided in the various embodiments of this specification may be used with each other. For example, the components of an embodiment having A, A′ and B and the components of an embodiment having A″, C and D can be used with each other in various combination, e.g., A, C, D, and, A, A″, C and D, etc., in accordance with the teaching of this specification. Thus, the scope of protection afforded the present inventions should not be limited to a particular embodiment, configuration or arrangement that is set forth in a particular embodiment, example, or in an embodiment in a particular Figure.

The invention may be embodied in other forms than those specifically disclosed herein without departing from its spirit or essential characteristics. The described embodiments are to be considered in all respects only as illustrative and not restrictive.

1. A system for the aggregation and enhancement of flare gas into an end product, the system comprising:
a. a plurality of gas-to-liquid (GTL) systems;
b. an initial liquid product enhancement (“IPE”) system;
c. wherein each of the GTL systems are located a distance from the IPE system;
d. wherein the GTL systems are in fluid communication with a flare gas source;
e. wherein GTL systems are configured to convert the flare gas into an initial liquid product;
f. a means for transporting the liquid initial product over each of the distances from each of the GTL systems to the IPE system; and,
g. the IPE system configured to convert the initial liquid product into a liquid end product.

2. The system of claim 1, wherein at least one of the distances from one of the GTL systems to the IP system is different than another of the distances from another of the GTL systems to the IPE system.

3. The system of claim 2, wherein the distances from each of the GTL systems to the IPL system are different.

4. The system of claim 1, wherein locations of the plurality of GTL systems defines an area, and the area is from about 0.5 miles² to about 10,000 miles².

5. The system of claim 1, comprising at least 20 GTL systems.

6. The system of claim 1, comprising at least 50 GTL systems.

7. The system of claim 1, comprising only a single IPE system.

8. The system of claim 1, wherein the initial liquid product comprises methanol.

9. The system of claim 1, wherein the end product comprises methanol.

10. The system of claim 1, wherein the initial liquid product comprises from about 50% to 95% methanol.

11. The system of claim 1, wherein the initial liquid product comprises methanol and has less than 1% CO₂.

12. The system of claim 1, wherein the initial liquid product comprises methanol having about 0.5 wt % CO₂.

13. The system of claim 1, wherein the liquid end product comprises at least 99.7% methanol.

14. The system of claim 1, wherein the liquid end product comprises at least 99.8% methanol.

15. The system of claim 1, wherein the liquid end product comprises at least 99.85% methanol.

16. The system of claim 1, wherein the liquid end product consists essentially of methanol, having less than 0.1 wt % water, less than 50 ppm (mg/kg) ethanol, and less than 30 ppm (mg/kg) acetone.

17. The system of claim 1, wherein the IPE comprises a distillation column.

18. The system of claim 1, wherein the IPE comprises a distillation column configured to remove water from the initial liquid product.

19. The system of claim 1, wherein IPE comprises a distillation column configured to remove water from the initial liquid product; and wherein the distillation column comprises a side stream.

20. The system of claim 1, wherein IPE comprises a distillation column configured to remove water from the initial liquid product; and wherein the distillation column comprises a dividing wall column.

21. The system of claim 1, wherein IPE comprises a distillation column configured to remove water from the initial liquid product; and wherein the distillation column comprises a dividing wall column and a side stream.

22. The system of claim 1, wherein IPE comprises a thermal junction, wherein the thermal junction comprises a universal heat-addition system.

23. The system of claim 1, wherein IPE comprises a universal heat-addition system.

24. The system of claim 1, wherein the means for transporting from at least one of the plurality of GTL systems comprises a truck, a rail car, a barge, a vessel, or a pipeline.

25. The system of claim 1 system of any of the forgoing claims, wherein at least one of the sources of flare gas is an oil well and the GTL is in fluid communication with the oilwell.

26. The system of claim 1 system of any of the forgoing claims, wherein at least one of the sources of flare gas is a wellhead and the GTL is in fluid communication with the wellhead.

27. The system of claim 1 system of any of the forgoing claims, wherein at least one of sources of flare gas is a wellhead; and wherein a conduit connects the GTL system to the wellhead, whereby the GTL system is in fluid communication with the wellhead.

28. The system of claim 1 system of any of the forgoing claims, wherein the IPE comprises a holding tank for receiving the initial liquid product from at least one of the plurality of GTL systems.

29. The system of claim 1 system of any of the forgoing claims, wherein the IPE is configured to remove water from the initial liquid product.

30. The system of claim 1 further comprising a control system, wherein the control system is in control communication with the plurality of GTL systems.

31. The system of claim 1 further comprising a control system, wherein the control system is in control communication with the plurality of GTL systems and the IPE system.

32. The system of claim 1 further comprising a control system, wherein the control system is in control communication with one or more of the plurality of GTL systems, the IPE system, or both.

33. The system of claim 1, wherein the GTL, the IPE or both are located off-shore.

34. A system for the aggregation and enhancement of flare gas into an end product comprising methanol, the system comprising:
a. a plurality of gas-to-liquid (GTL) systems, wherein the GTL systems are in fluid communication with a plurality of sources of a flare gas to thereby provide the flare gas to the GTL systems; wherein the GTL systems are configured to convert the flare gas into an initial methanol;
b. an initial liquid product enhancement (“IPE”) system; wherein the IPE comprises a distillation column configured to remove water from the initial methanol, to thereby provide an end product methanol having at least 98% methanol; and,
c. a means for conveying the initial methanol from each of the GTL systems to the IPE system.

35. The system of claim 34, wherein the source of the flare gas is a wellhead.

36. The system of claim 34, wherein the plurality of GTL systems comprises one or more onsite GTL systems; wherein the plurality of sources of flare gas comprises one or more wellheads; and wherein each of the plurality of onsite GTL systems is in fluid communication with only one wellhead.

37. The system of claim 34, wherein the plurality of GTL systems comprises onsite GTL systems; wherein the plurality of sources of flare gas comprises wellheads; and wherein at least one of the plurality of onsite GTL systems is in fluid communication with only one wellhead.

38-68. (canceled)

69. A method for the aggregation and enhancement of flare gas into an end product, the method comprising:
a. placing a plurality of gas-to-liquid (GTL) systems at a plurality of locations to thereby define a GTL location for each of the plurality of GTL system;
b. placing an initial liquid product enhancement (“IPE”) system at an IPE location; wherein each of the GTL systems is a distance from the IPE system;
c. receiving a flow of a flare gas into one or more of the GTL systems;
d. wherein the GTL systems receiving the flare gas converts the flare gas into an initial liquid product; and,
e. receiving the initial liquid product from one or more of the GTL systems at the IPE system; and,
f. the IPE system converting the initial liquid product into a liquid end product.

70. The method of claim 69, wherein the initial liquid product comprises from about 25% to about 90% methanol.

71. (canceled)

72. (canceled)

73. (canceled)

74. (canceled)

75. (canceled)

76. (canceled)

77. (canceled)

78. (canceled)

79. (canceled)

80. (canceled)

81. A method for the aggregation and enhancement of flare gas into an end product, the method comprising:
a. receiving a flare gas into an onsite gas-to-liquid (GTL) system;
b. converting the received flare gas into an initial methanol;
c. receiving the initial methanol into an initial liquid product enhancement (“IPE”) system, wherein the IPE system is located at least 0.5 miles away from a location off the GTL system; and,
d. converting the initial methanol into end product methanol having at least 98.5% methanol.

82. The method of claim 81, wherein the initial methanol comprises from about 70% to 95% methanol.

83. (canceled)

84. (canceled)

85. (canceled)

86. (canceled)

87. (canceled)

88. (canceled)

89. (canceled)

90. (canceled)

91. A method for the aggregation and enhancement of flare gas into an end product, the method comprising:
a. receiving a flare gas into one or more of a plurality of onsite gas-to-liquid (GTL) system;
b. converting the received flare gas into an initial methanol, wherein the initial methanol comprises crude methanol, stabilized methanol or both;
c. wherein the composition of the initial methanol varies over time, varies between two or more of the GTL systems, or both;
d. receiving the initial methanol from one or more of the plurality of GTL systems at a hub; wherein the hub comprises a tank and an initial liquid product enhancement (“IPE”) system;
e. blending the initial methanol from at least one of the GTL systems with the initial methanol from at least another of the GTL systems, to thereby provided a blended initial methanol;
f. wherein the IPE system is located at least 0.5 miles away from a location off one or more of the GTL systems; and,
g. converting the blended initial methanol into an end product methanol having at least 98.5% methanol.

92-99. (canceled)
